SEQUENCE LISTING (10 sequences)

SEQ ID NO: 1 - protein, 5 aa, synthetic construct
SYVMH

SEQ ID NO: 2 - protein, 6 aa, synthetic construct
NPYNDG

SEQ ID NO: 3 - protein, 12 aa, synthetic construct
GTYYYGTRVFDY

SEQ ID NO: 4 - protein, 16 aa, synthetic construct
RSSKSLQNVNGNTYLY

SEQ ID NO: 5 - protein, 7 aa, synthetic construct
RMSNLNS

SEQ ID NO: 6 - protein, 9 aa, synthetic construct
MQHLEYPIT

SEQ ID NO: 7 - protein, 121 aa, synthetic construct
EVQLVESGGGLVKPGGSLKLSCAASGYTFTSYVMHWVRQAPGKGLEWIGYINPYNDGTKYNEKFQGRVTISSDKSISTAYMELSSLRSEDTAMYYCARGTYYYGTRVFDYWGQGTLVTVSS

SEQ ID NO: 8 - protein, 112 aa, synthetic construct
DIVMTQSPATLSLSPGERATLSCRSSKSLQNVNGNTYLYWFQQKPGQSPQLLIYRMSNLNSGVPDRFSGSGSGTEFTLTISSLEPEDFAVYYCMQHLEYPITFGAGTKLEIK

SEQ ID NO: 9 - protein, 329 aa, synthetic construct
ASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPDVFLFPPKPKDTLMISRTPEVTCVVVDVSHEDPEVQFNWYVDGVEVHNAKTKPREEQFNSTFRVVSVLTWHQDWLNGKEYKCKVSNKALPAPEEKTISKTKGQPREPQVYTLPPSREEMTKNQVSLTCLVKGFYPSDIAVEWESNGQPENNYKTTPPMLDSDGSFFLYSKLTVDKSRWQQGNVFSCSVMHEALHNHYTQKSLSLSPGK

SEQ ID NO: 10 - protein, 106 aa, synthetic construct
RTVAAPSVFIFPPSDEQLKSGTASWCLLNNFYPREAKVQWKVDNALQSGNSQESVTEQDSKDSTYSLSSTLTLSKADYEKHKVYACEVTHQGLSSPVTKSFNRGEC

(19) United States
(12) Patent Application Publication
(10) Pub. No.: US 2024/0360215 A1
(43) Pub. Date: Oct. 31, 2024

(54) ANTI-CD19 ANTIBODY IN THE TREATMENT OF CNS LYMPHOMA

(71) Applicant: The Regents of the University of California, Oakland, CA (US)

(72) Inventor: James L. Rubenstein, San Francisco, CA (US)

(21) Appl. No.: 18/645,760

(22) Filed: Apr. 25, 2024

Related U.S. Application Data
(60) Provisional application No. 63/498,789, filed on Apr. 27, 2023.

(51) Int. Cl. C07K 16/28; A61K 9/00; A61K 31/454; A61K 31/519; A61K 39/00; A61P 35/00

(52) U.S. Cl. CPC C07K 16/2803; A61K 9/0019; A61K 31/454; A61K 31/519; A61P 35/00; A61K 2039/505; A61K 2039/54; A61K 2039/545; C07K 2317/24

(57) ABSTRACT

The disclosure provides a method of treating CNS lymphomas using a combination therapy comprising an anti-CD19 antibody and an immunomodulatory imide drug.

### CROSS-REFERENCE TO RELATED APPLICATIONS

This application claims priority benefit of U.S. Provisional Application No. 63/498,789, filed Apr. 27, 2023, which is incorporated by reference for all purposes.

### STATEMENT AS TO RIGHTS TO INVENTIONS MADE UNDER FEDERALLY SPONSORED RESEARCH AND DEVELOPMENT

This invention was made with government support under R01 CA139083 awarded by the National Institutes of Health. The government has certain rights in the invention.

### SEQUENCE LISTING

The instant application contains a Sequence Listing which has been submitted electronically in XML format and is hereby incorporated by reference in its entirety. Said XML copy, created on May 20, 2024, is named 081906-1431462-260810US_SL.xml and is 10,356 bytes in size.

### BACKGROUND

The CD19 transmembrane glycoprotein is expressed by B cells throughout most stages of differentiation by the majority of B-cell malignancies and by follicular dendritic cells. Given the high incidence of neurotoxicity associated with blood-brain barrier disruption with CD19-directed chimeric antigen receptor (CAR)-T cells and bispecific T-cell engager antibodies (Gust et al, Cancer Discov. 7:1404-19, 2017; Goebeler et al, J. Clin Oncol. 34:1104-11, 2016; Nagorsen, et al., Pharmaco. Ther. 136:334-42, 2012; Neelapu et al, Hematol Oncol. 37(Suppl 1): 48-52, 2019), Parker, et al, Cell 183:126-142:e117, 2020 recently investigated the possibility of CD19 expression by non-B cells in the brain. Using single-cell RNA sequencing data generated from human brain, they identified CD19 expression by brain mural cells or pericytes, an integral component of the neurovascular unit that regulates integrity of the blood-brain barrier (Armulik et al, Nature 468:557-61, 2010). CD19 expression by mural cells was confirmed in additional datasets and by immunohistochemistry, and was found along both small capillaries and larger vessels. Brain mural cell expression of CD19 also exhibited regional variation, with higher expression in the hippocampus, insula, temporal, frontal and parietal lobes. Finally, it was demonstrated that brain mural cell expression of CD19 is conserved in mice and that the infusion of CAR-T cells targeting murine CD19 into non-obese diabetic severe combined immunodeficiency (NOD/SCID) gamma (NSG) mice, lacking B cells, resulted in increased blood-brain barrier permeability and mural cell depletion (Parker, 2020, supra). These results, therefore, provide a potential on-target mechanism for CD19 CAR-T cell mediated neurotoxicity.

### BRIEF SUMMARY

The Summary is provided to introduce a selection of concepts that are further described below in the Detailed Description. This Summary is not intended to identify key or essential features of the claimed subject matter, nor is it intended to be used as an aid in limiting the scope of the claimed subject matter.

In the present disclosure, tafasitamab, an anti-CD19 monoclonal antibody, is identified as a therapeutic agent for treatment of CNS lymphomas, e.g., refractory CNS in immunocompetent patients. Not to be bound by theory, such treatment has potential to modulate blood-brain barrier integrity by perturbation of CD19 expressed by mural cells, but without concomitant cytokine release syndrome, given that tafasitamab does not recruit a cytotoxic payload of T cells.

In some aspects, provided herein is a method of treating a central nervous system (CNS) lymphoma in an immunocompetent subject, the method comprising administering a therapeutically effective amount of tafasitamab and a immunomodulatory imide drug (IMiD) to the subject. In some embodiments, the IMiD is lenalidomide or pomalidomide. In some embodiments, lenalidomide is administered orally in an amount from 5 mg/day to 35 mg/day. In some embodiments, lenalidomide is administered orally in an amount from 25 mg/day to 30 mg/day. In some embodiments, the method comprises administering tafasitamab intravenously in a dose range of 12 mg/kg to 48 mg/kg. In certain embodiments, tafasitamab is administered at a dose of 12 mg/kg. In some embodiments, the method comprises administering tafasitamab at least twice week for at least one month. In some embodiments, tafasitamab is administered at least twice a week for two or more months. In certain embodiments, tafasitamamb at least once a month for subsequent months of treatment. In some embodiments, tafasitamab is administered on days 1, 4, 8, 15, and 22 of at least one 28-day cycle followed by administration of tafasitamab on days 1, 8, 15, and 22 of a second 28-day cycle followed by administration of tafasitamab on days 1 and 5 of one or more subsequent 28-day cycles. In some embodiments, tafasitamab is administered intraventicularly by lumbar puncture. In certain embodiments, tafasitamab is administered with an Ommaya reservoir.

In another aspect, the disclosure provides a method of treating a central nervous system lymphoma in an immunocompetent subject, the method comprising administering a therapeutically effect amount of tafasitamab and a Bruton's tyrosine kinase (BTK) inhibitor to the subject. In certain embodiments, the BTK inhibitor is ibrutinib. In some embodiments, the method comprises administering tafasitamab by intravenous infusion in a dose range of 12 to 48 mg/kg. In some embodiments, tafasitamab is administered at a dose of 12 mg/kg. In some embodiments, tafasitamab is administered at least twice week for at least one month. In certain embodiments, tafasitamab is administered at least twice a week for two or more months. IN some embodiments, the method comprises administering tafasitamamb at least once a month for subsequent months of treatment. In some embodiments, tafasitamab is administered on days 1, 4, 8, 15, and 22 of at least one 28-day cycle followed by administration of tafasitamab on days 1, 8, 15, and 22 of a second 28-day cycle followed by administration of tafasitamab on days 1 and 5 of one or more subsequent 28-day cycles. In some embodiments, ibrutinib is administered orally in an amount from 300 mg to 700 mg/day. In certain embodiments, ibrutinib is administered orally in an amount of 400 mg/day to 600 mg/day. In some embodiments, tafasitamab is delivered intraventicularly by lumbar puncture. In certain embodiments tafasitamab is administered with an Ommaya reservoir.

In some embodiments of the methods provided by the present disclosure, the CNS lymphoma is a primary lymphoma. In some embodiments, the CNS lymphoma is a secondary lymphoma. In some embodiments, the patient has resistance to lenalidomide, pomalidomide, and/or ibrutinib.

In some embodiments, the CNS lymphoma is an intraocular lymphoma. In certain embodiments, the CNS lymphoma is an ocular lymphoma and tafasitamab is delivered by intraocular or intravitreal injection. In some embodiments, tafasitamab is administered in an amount of about 0.1-0.5 mg/ml. In some embodiments, tafasitamab is administered once a month.

In some embodiments, tafasitamab is administered in combination with another therapeutic agent that targets Cereblon (CRBN), e.g., an immunomodulatory imide drug (IMiD) such as a thalidomide analog.

In other aspects, an anti-CD-19 antibody as described herein, e.g., tafasitimab can be used alone or in combination with another pharmaceutical compound, e.g., an IMiD, in a patient with systemic CD19 lymphoid malignancies who is at risk for CNS lymphoma, to prevent or redue the risk of secondary CNS lymphoma. In some embodiments, an anti-CD-19 antibody as described herein, e.g., tafasitimab, can be used in combination with intrathecal methotrexate or high-dose methotreaxate to reduce the risk of second CNS lyimphoma in patients otherwise at high risk.

### BRIEF DESCRIPTION OF DRAWINGS

FIG. 1: CSF flow cytometry (upper panel) and cytology (lower panel) from Patient #2 (left, pre-treatment; right, after 1 month on treatment with tafasitamab+lenalidomide 10 mg/day) demonstrating clearance of lambda-restricted B-cells from the CSF. This patient's disease previously progressed on lenalidomide monotherapy at a higher dose of 15 mg/day.

### DETAILED DESCRIPTION

As used herein, “a”, “an”, and “the” include aspects with one member, but also include aspects with more than one member unless the context clearly dictates otherwise.

The terms “about” and “approximately” as used herein with respect to a given value generally mean a deviation from the stated value that is typically within 30%, preferably within 20% of the stated value. For example, “about” with respect to doses or amounts is recognized by one of ordinary skill in the art to provide a pharmacological effect equivalent to that obtained from the specified dose or amount. In certain embodiments, the terms “about” and “approximately,” when used in this context, includes a dose or amount within 30%, within 20%, within 15%, within 10%, or within 5%, of the specified dose or amount.

As used herein, the term “treat” or any grammatical variation thereof (e.g., treat, treating, treatment, etc.) refers to a therapeutic treatment of a disease in a subject, as well as prophylactic or preventative measures towards the disease. A therapeutic treatment can slow the progression of the disease, ameliorate disease symptoms, improve the subject's outcome, eliminate the disease, and/or alleviate, reduce or eliminate the symptoms of the disease. Thus, beneficial or desired clinical results include, but are not limited to, alleviation of disease symptoms, diminishment of the extent of the disease, stabilization (i.e., not worsening) of the disease, inhibiting, suppressing, or delaying or slowing disease progression, or amelioration of the disease state. Those in need of treatment include those already having the disease, condition, or disorder, as well as those at high risk of having the disease, condition, or disorder, and those in whom the disease, condition, or disorder is to be prevented.

As used herein and unless otherwise indicated, the term “preventing” means a method of delaying and/or precluding the onset, recurrence or spread, in whole or in part, of a disorder, disease or condition, such a dissemination of a high grade lymphoma to the brain.

### Tafasitamab

The term “tafasitamab” is synonymous with “MOR0208” and “XmAb5574” and refers to a humanized, Fc-modified, CD19-targeting antibody having a full length heavy chain amino acid sequence and full-length light chains sequences shown in SEQ ID NO:11 and SEQ ID NO:12, respectively of WO2022/117799. See, also, U.S. Pat. No. 8,524,867. Tafasitumab includes the U.S. Food and Drug Administration (FDA) approved Monjuvi® (tafasitumab-cxix).

In some embodiment, an anti-CD19 antibody administered with a compound, e.g., an immunomodulatory imide drug (IMiDs), such as lenalidomide or pomalidomide, in accordance with the present disclosure comprises a heavy chain variable region comprising an HCDR1 comprising SYVMH (SEQ ID NO: 1), an HCDR2 comprising NPYNDG (SEQ ID NO: 2), and an HCDR3 comprising GTYYYGTRVFDY (SEQ ID NO: 3); and a light chain variable region comprising an LCDR1 comprising RSSKSLQNVNGNTYLY (SEQ ID NO: 4), an LCDR2 comprising RMSNLNS (SEQ ID NO: 5), and an LCDR3 region comprising MQHLEYPIT (SEQ ID NO: 6). In some embodiments, an anti-CD19 antibody administered in accordance with the present disclosure comprises a heavy chain variable region sequence comprising EVQLVESGGGLVKPGGSLKLSCAASGYTFTSYVMHWVRQAPGKGLEWIGYI NPYNDGTKYNEKFQGRVTISSDKSISTAYMELSSLRSEDTAMYYCARGTYYY GTRVFDYWGQGTLVTVSS (SEQ ID NO: 7) and a light chain variable region sequence comprising DIVMTQSPATLSLSPGERATLSCRSSKSLQNVNGNTYLYWFQQKPGQSPQL LIYRMSNLNSGVPDRFSGSGSGTEFTLTISSLEPEDFAVYYCMQHLEYPITFGAGTKLEIK (SEQ ID NO: 8). In some embodiments, such an anti-CD19 antibody has an Fc region comprising a mutation that enhances effector function, e.g., enhances ADCC, relative to a naturally occurring human Fc region. In some embodiments, the antibody comprises an Fc domain comprising an amino acid substitution at position S239 and/or I332 (EU numbering scheme). In some embodiments, the anti-CD19 antibody comprises a heavy chain constant region sequence ASTKGPSVFPLAPSSKSTSGGTAALGCLVKDYFPEPVTVSWNSGALTSGVHTFPAVLQSS GLYSLSSVVTVPSSSLGTQTYICNVNHKPSNTKVDKKVEPKSCDKTHTCPPCPAPELLGG PDVFLFPPKPKDTLMISRTPEVTCVVVDVSHEDPEVQFNWYVDGVEVHNAKTKPREEQF NSTFRVVSVLTWHQDWLNGKEYKCKVSNKALPAPEEKTISKTKGQPREPQVYTLPPSRE EMTKNQVSLTCLVKGFYPSDIAVEWESNGQPENNYKTTPPMLDSDGSFFLYSKLTVDKS RWQQGNVFSCSVMHEALHNHYTQKSLSLSPGK (SEQ ID NO: 9) and a light chain constant region sequence

(SEQ ID NO: 10)
RTVAAPSVFIFPPSDEQLKSGTASWCLLNNFYPREAKVQWKVDNALQSG
NSQESVTEQDSKDSTYSLSSTLTLSKADYEKHKVYACEVTHQGLSSPVT
KSFNRGEC.

### Pharmaceutical Compounds

In some embodiments, pharmaceutical agents that can be used in combination with tafasitamab, or alternative anti-CD19 antibody, e.g., as described herein in the preceding paragraph, to treat CNS lymphoma are compounds that modulate the Cereblon-E3 ubiquitin ligase complex. Such compounds include immunomodulatory imide drugs (IMiDs) that bind Cereblon (CRBN), which is the substrate receptor of an E3 ubiquitin ligase complex. When bound to IMiDs, cereblon is able to target a number of substrates involved in cancer cell growth and proliferation for degradation. Illustrative compounds that target cereblon include IMiDs, such as, but not limited to, lenalidomide, pomalidomide, avadomide (CC-122), iberdomide (CC-220), CC-885, CC-90009 (eragidomide), CC-99282 (CAS No. 2379572-34-4), CC-91633 (BMS-986397), and mezigdomide (CC-92480) (see, e.g., WO2021/222552, WO2022/053652, WO2023/057893, Nature 535: 252-7, 2016, each incorporated by reference) or a pharmaceutically acceptable salt thereof. IMiDs typically contain a glutarimide ring that interacts with a conserved hydrophobic pocket of cereblon and a variable phthaloyl ring (see, e.g., Nat. Struct Mol Biol 21:803-8098, 2014; Nature 523:183-8, 2015; Nature 532:127-30, 2016). In some embodiments, the compound has an extended struct that includes phenyl and morpholino moieties that enhance interactions with CRBN or substrates (see, e.g., Oncotarget, 12:1555-1563, 2021). Additional agents that target and bind to cereblon are described, for example in WO2022/032132). In some embodiments, an agent administered in accordance with the disclosure is lenalidomide, pomalidomide, iberdomide, avadomide or mezigdomide. In some embodiments, the therapeutic agent is a thalidomide derivative such as lenalidomide or an analog thereof (see, e.g., U.S. Pat. Nos. 5,635,517; 6,555,554; 7,119,106; 7,465,800; 7,855,217; 8,288,415; 8,530,498, or 8,822,499; or 9,139,556) or pomalidomide or an analog thereof (see, e.g., U.S. Pat. Nos. 5,635,517; 6,316,471; 6,476,052; 8,158,653; 8,198,262;8,673,939; 8,735,428; and 8,828,427).

In other embodiments, a Bruton's tyrosine kinase (BTK) inhibitor can be used in combination with tafasitimab, or an alternative anti-CD19 antibody as described herein, to treat a CNS lymphoma. In some embodiments, the BTK inhibitor is ibrutinib, acalbrutinib, or zaubrutinib (see, e.g., Wen et al, Leukemia 25:312-332, 2021). Additional illustrative BTK inhibior include GDC-0834, RN-486, CGI-560, CGI-1764, HM-71224, CC-292, ONO-4059, CNX-774, or LFM-A13 (see, e.g., J. Hematology & Oncol. 6:59, 2013).

### Administration of Combination Tafasitamab and a Pharmaceutical Compound

### Administration of Tafasitimab

Tafasitamab, or an alternative anti-CD-19 antibody as described herein, may be administered by any suitable route to treat a target CNS lymphoma. In some embodiments, tafasitimab is administered intravenously to treat the CNS lymphoma.

In some embodiments, tafasitamab is administered by intravenous infusion in a dose range of 10 mg/kg to 50 mg/kg. In some embodiments, tafasitamab is administered by intravenous infusion in a dose range of 12 mg/kg to 48 mg/kg. In some embodiments, tafasitamab is administered at a dose of 10-15 mg/kg. In some embodiments, tafasitamab is administered at a dose of 12 mg/kg.

Tafasitimab can be administered at regular intervals, e.g., over multiple months of treatment. For example, in some embodiments, tafasitamab is administered intravenously at least twice a week for at least one month. In some embodiments, tafasitamab is administered at least twice a week for two or more months. In some embodiments, tafasitamamb is administered at least once a week for at least one month. In some embodiments, a patient may be treated with tafasitamab for a number of months in which tafasitamab is initially administered twice a week when treatment is initiated followed by administration one day a week or one day every two weeks.

In some embodiments, tafasitamab is administered intravenously on days 1, 4, 8, 15, and 22 of at least one 28-day cycle followed by administration on days 1, 8, 15, and 22 of a second 28-day cycle followed by administration of tafasitamab on days 1 and 15 of one or more subsequent 28-day cycles.

In some embodiments, tafasitamab, or an alternative anti-CD-19 antibody as described herein, is delivered intrathecally, e.g., intraventicular administration by lumbar puncture. In some embodiments, delivery employs an Ommaya reservoir ventricular access device for repetitive access to the intrathecal space for delivery of the antibody. In some embodiments, tafasitamab is administered by intrathecal or intraventricular administration at a dose of about 10 mg to about 50 mg, or a range of about 10 mg to about 35 mg.

In some embodiments in which the CNS lymphoma is an intraocular lymphoma, tafasitamab, or an alternative anti-CD-19 antibody as described herein, is administered by injection into the eye, e.g., intravitreal injection. In some embodiments, tafasitamab is administered at a concentration of 0.5-2.0 mg/ml, e.g., at a concentration of 1 mg/ml. In some embodiments, tafasitamab is injected two times a month, once a month, or once every two months, or at greater intervals. In some embodiments tafasitimab is initially administered once the first month of treatment followed by administration every two months, depending on response of the tumor to treatment with the combination therapy.

### Administration of Pharmaceutical Compound

### IMiD Compounds

A pharmaceutical compound, e.g., a IMiD such as lenalidomide or pomalidomide is typically administered orally at a frequency of one dose a day. In some embodiments, the IMID is administered a dose of from administered at a dose of about 5 mg, 10 mg, 20 mg, 25 mg, or 30 mg/day. In some embodiments, lenalidomide or pomalidomide is administered for the first five days, the first seven days, or the first ten days of a 28-day cycle, for example, at a dose of about 25 mg/day, 20 mg/day, 15 mg/day, 10 mg/day or 5 mg/day on days 1-21 of each cycle. In some embodiments, pomalidomide is administered in an amount of about 3 mg to 5 mg/day.

In some embodiments, e.g., in cases here the IMiD has an extended structure that includes phenyl and morpholino moieties that enhance interactions with cereblon, the amount of IMiD administered is less than lenalidomide or pomalidomide. For example, in some embodiments, iberdomide is administered in an amount of about 0.1 mg to about 2 mg per day. In some embodiments, avadomide is administered in an amount of about 2 mg to about 50 mg per day. In some embodiments, mezigdomide is administered in an amount of about 0.1 mg to about 2 mg/day.

In some embodiments, lenalidomide, pomalidomide, avadomide iberdomid or mezigdomide is administered the first five days, the first seven days, or the first ten days of a 28-day cycle.

### BTK Inhibitor Compounds

In some embodiments, a subject having a CNS lymphoma is administered tafasitimab, or an alternative CD-19 antibody as described herein, in combination with a BTK inhibitor, such as ibrutinib, acalbrutinib, or zaubrutinib. In some embodiments, the BTK inhibitor is ibrutinib.

In some embodiments, ibrutinib is administered orally in an amount of about 100 mg, 200 mg, 300, mg, 400 mg, 500 mg, 600 mg, or 700 mg/day. In some embodiments, ibrutinib is administered daily at a dose of 280 mg/day, 420 mg/day, or 560 mg/day.

In some embodiments, a patient is administered 12 mg of tafasitamab intravenously on days 1, 4, 8, 15, and 22 of a first 28-day cycle; days 1, 8, 15, and 22 of a second and third 28-day cycle; and days 1 and 15 of subsequent 28-days cycles; along with lenalidomide on each day of the first 21 days of each cycle, e.g., at a dose of 5, 10, or 15 mg/day; or at a dose of 20 or 30 mg/day.

The combination of tafasitamab, or alternative anti-CD19 monoclonal antibody, is administered to achieve a therapeutic effect, for example, at least a partial response, e.g., in which the size of the tumor decreases.

### Other Therapeutic Compounds

In some embodiments, tafasitimab is administered in conjunction with another therapeutic agent for the treatment of a neurological condition or disorder to facilitate CNS penetration of the therapeutic agent. For example, tafasitimab may be used in combination with a drug to treat a brain tumor, e.g., glioblastoma, a neurodegenerative disease, dementia, or a psychiatric disease. Examples of such diseases include, but are not limited to, Alzheimer's Disease, frontotemporal dementia, dementia with Lewy bodies, Huntington's Disease, Parkinson's Disease, glioblastoma or neuroblastoma, multiple sclerosis, chronic inflammatory demyelinating polyneuropathy, amyotrophic lateral sclerosis, or any autoimmune disease with neurologic involvement or neuromuscular disease. In some embodiments, tafasitamab, or an alternative anti-CD19 monoclonal antibody as described herein, is used to treat multiple sclerosis.

### CNS Lymphomas

In some embodiments, the compounds described herein may be used in the treatment or management of any CNS lymphoma including primary and secondary CNS lymphomas. Such lymphomas include primary intraocular lymphomas and secondary intraocular lymphomas. Intraocular lymphomas can arise in the retina, uvea, vitreous, Bruch's member and optic nerve.

In a further aspect of the disclosure, an anti-CD-19 antibody as described herein, e.g., tafasitimab, can be used alone or in combination with another pharmaceutical compound, e.g., an IMiD such as lenalidomide or pomalidomide, to treat a patient with systemic CD19 lymphoid malignancies who is at risk for CNS lymphoma, to prevent or reduce the risk of secondary CNS lymphoma. In some embodiments, an anti-CD-19 antibody as described herein, e.g., tafasitimab, Thus, in some embodiments, tafasitamab and an IMiD such as lenalidomide or pomalidomide can be administered to a patient with a high grade lymphoma to inhibit dissemination to other tissues. In some embodiments, an anti-CD-19 antibody as described herein, e.g., tafasitimab, can be used in combination with intrathecal methotrexate or high-dose methotrexate to reduce the risk of second CNS lymphoma in patients otherwise at high risk.

In some embodiments, a patient having a CNS lymphoma, such as a large B-cell-type lymphoma, may have relapsed following treatment with a previous therapeutic regimen. In some embodiments, the previous therapeutic regimen may have included an IMiD, such as lenalidomide or pomolidomide; or a BTK inhibitor such as ibrutinib. Thus, in some embodiments, the patient may have at least partial resistance to the IMID or BTK inhibitor that is administered with trafasitamab, or alternative anti-CD19 monoclonal antibody, in accordance with the present disclosure.

### Illustrative Treatment of Two Patient Having CNS Lymphomas

Tafasitamab and lenalidomide were evaluated in a phase II L-MIND study (Clinical Trials (Clinical trials.gov; Identifier: NCT02399085). It was observed that tafasitamab plus lenalidomide exhibited synergistic efficacy in relapsed/refractory aggressive non-Hodgkin lymphoma, with an overall response rate of 60%, complete response rate of 43%, and a favorable toxicity profile compared to CAR-T, without neurotoxicity. Notably, the L-MIND trial excluded patients previously treated with immunomodulatory drugs as well as CNS lymphoma (Salles et al, Lancet Oncol. 21:978-88, 2020)

The first patient was a 53 year-old man who presented with seizure and a right frontal lobe mass that upon pathological analysis revealed large-B cell lymphoma, activated B-cell type. Staging documented concomitant right adrenal involvement. Treatment at an outside institution consisted of six cycles of high-dose methotrexate (HD-MTX) intercalated with rituximab, cyclophosphamide, adriamycin, vincristine, prednisone (R-CHOP) plus intrathecal cytarabine, also six cycles. At 7 months there was multifocal CNS progression and the patient was transferred to a tertiary care center where he received eight cycles of HD-MTX, six doses of temozolomide plus rituximab (MTR), consolidation with etoposide-ara-C (Soussain et al, J. Clin. Oncol 19:742, 9, 20021; Rubenstein et al, J. Clin Oncol. 31:3061-8, 2013) followed by autologous stem cell transplantation and lenalidomide maintenance. However, at 6 months after transplantation, multifocal CNS progression was again evident on magnetic resonance imaging (MRI). This second relapse was treated with repeat HD-MTX (eight cycles) plus rituximab followed by focal irradiation to a refractory lesion in the right postcentral gyrus, followed by pomalidomide maintenance, resulting in a CNS response duration of nearly 2 years. A third isolated CNS relapse manifested by new multifocal lesions with mild left-to-right midline shift. Sequential repeat HD-MTX plus rituximab (three cycles), gamma knife radiation, ibrutinib monotherapy (with isavuconazonium) and ibrutinib plus lenalidomide (10 mg/day) yielded only a partial response on MRI.

Severe treatment-related toxicities requiring inpatient management during this phase included two independent episodes of pneumonia, associated with respiratory syncytial virus and later, aspergillosis, during pomalidomide, and pancytopenia, gastrointestinal bleeding, and necrotizing gangrenous cholecystitis, during treatment with ibrutinib and ibrutinib/lenalidomide.

Resistance to ibrutinib plus lenalidomide was ultimately manifest by growth of the enhancing nodules involving the left anterior temporal lobe and right posterior cingulate gyrus; these exhibited restricted diffusion consistent with CNS lymphoma progression. Ibrutinib was therefore discontinued and the patient continued on lenalidomide (10 mg/day), now co-administered with intravenous tafasitamab (12 mg/kg), without dexamethasone, following the schedule of the L-MIND study. By 2 months of therapy, the CNS lesions exhibited partial regression. Near complete resolution of lesional enhancement on MRI was achieved by 6 months (for image, see, Rauschecker et al., Br J Haematol, 201:154-157, first published Jan. 23 2023). The patient continues twice monthly infusion of tafasitamab, plus lenalidomide days 1-21, and remains in radiographic remission, both in brain and systemically, at 22 months since starting this combination. Notably the duration of complete MRI response during tafasitamab/lenalidomide was greater than three-times longer than with lenalidomide monotherapy after transplant and only partial responses on MRI were achieved with ibrutinib and ibrutinib plus lenalidomide. Tafasitamab plus lenalidomide has been tolerated well, without infectious complications or evidence of neurotoxicity.

The second patient is an 82-year-old woman who presented with multifocal primary CNS lymphoma, large B-cell type, who achieved a complete response on MRI with MTR, followed by lenalidomide maintenance (5 mg/day, days 1-21) with a response duration of 17 months (Vu et al, Br J Haematol. 186:180-3, 2019). At first relapse she was re-treated with MTR followed by lenalidomide maintenance (5 mg/day) with intermittent high-dose methotrexate/rituximab every 4 months, resulting in sustained disease control on MRI for 3 years. At progression on lenalidomide she was re-challenged with MTR but immediately experienced growth of new lesions in the left frontal lobe, centrum semiovale and splenium of the corpus callosum. Tafasitamab plus lenalidomide (5 mg/day) was administered, without dexamethasone, and resulted in complete resolution of the two left hemispheric lesions. However, the corpus callosal lesion was resistant. In this case, MRI perfusion-weighted imaging (Am J Neuroradio. 34-35-40, 2013) was also performed and demonstrated elevated vascular perfusion solely in the two responding lesions. (For image, see, Rauschecker et al., Br J Haematol, 201:154-157, first published January 23 2023).

These illustrative treatments provide evidence for the activity of tafasitamab plus lenalidomide in primary and secondary CNS lymphomas. The role of tafasitamab in these cases is evident based on prior resistance to lenalidomide, pomalidomide and rituximab. Given these results, the absence of neurotoxicity, and the recognition of CD19 as a component of the blood-brain barrier, tafasitamab can have unique potential in CNS lymphomas, including as prophylaxis in selected cases (Orellana-Noia et al, Blood 139:413-23, 2022; Mckay et al, Br J Haematol 190-708-714, 2020). A phase I/II study, NCT05351593, has been developed to further evaluate tafasitamab plus lenalidomide in relapsed CNS lymphomas.

### Phase 1 Trial

Thus far, four patients have participated in the phase I trial of tafasitamab plus lenalidomide in relapsed CNS lymphoma. Three were treated at the 10 mg lenalidomide dose level and one at the 15 mg lenalidomide dose level. There have been no significant or serious adverse events to date. Two of the three patients treated at the lenalidomide 10 mg dose level responded to the combination. The longest response duration was six months. The patient treated at the 15 mg lenalidomide dose level has had stable disease in the CNS thus far, one month into therapy.

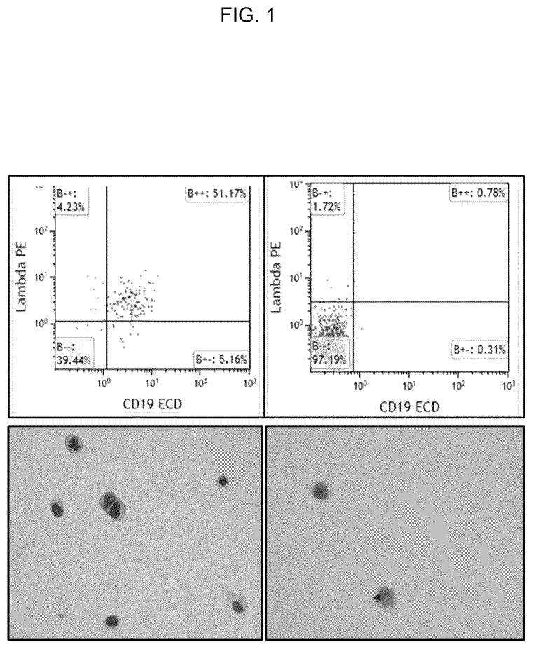

Illustrative responses of patient 2 in this study are depicted in FIG. 1. Patient 2 of the study, whose disease was previously refractory to lenalidomide 15 mg/day (off study) as well as refractory to high-dose methotrexate plus rituximab, administered one month previously, showed a complete response demonstrated by flow-cytometry (upper panel) in the leptomeningeal compartment with resolution of CD19+ malignant lymphoma cells in the cerebrospinal fluid within one month of treatment with lenalidomide 10 mg/day plus intravenous tafasitamab. The complete response was confirmed by CSF cytology (lower panel). Given the prior resistance to rituximab, these results are indicative that tafasitamab has enhanced activity in the CNS, perhaps with better CSF penetration compared to rituximab.

Each reference, publication, and citation provided herein is incorporated by reference for the purpose for which it was cited to the same extent as if each reference was individually incorporated by reference.

1. A method of treating a central nervous system (CNS) lymphoma in an immunocompetent subject, the method comprising administering a therapeutically effective amount of tafasitamab and an immunomodulatory imide drug (IMiD) to the subject.

2. The method of claim 1, wherein the IMiD is lenalidomide or pomalidomide.

3. (canceled)

4. The method of claim 1, wherein tafasitamab is administered intravenously in a dose range of 12 mg/kg to 48 mg/kg or at a dose of 12/mg/k; and/or the IMiD is lenalidomide and lenalidomide is administered orally in an amount from 5 mg/day to 35 mg/day or is administered orally in an amount from 25 mg/day to 30 mg/day.

5.-7. (canceled)

8. The method of claim 1, comprising administering tafasitamab at least twice week for at least one month or at least twice a week for two or more months, optionally further comprising administering tafasitamab at least once a month for subsequent months.

9.-10. (canceled)

11. The method of claim 1, wherein tafasitamab is administered on days 1, 4, 8, 15, and 22 of at least one 28-day cycle followed by administration of tafasitamab on days 1, 8, 15, and 22 of a second 28-day cycle followed by administration of tafasitamab on days 1 and 5 of one or more subsequent 28-day cycles.

12. The method of claim 1, where tafasitamab is administered intraventicularly by lumbar puncture, optionally wherein tafasitamab is administered with an Ommaya reservoir.

13. (canceled)

14. A method of treating a central nervous system lymphoma in an immunocompetent subject, the method comprising administering a therapeutically effect amount of tafasitamab and a Bruton's tyrosine kinase (BTK) inhibitor to the subject.

15. The method of claim 14, wherein the BTK inhibitor is ibrutinib.

16. The method of claim 14, comprising administering tafasitamab by intravenous infusion in a dose range of 12 to 48 mg/kg or at a dose of 12 mg/kg.

17. (canceled)

18. The method of claim 14, comprising administering tafasitamab at least twice week for at least one month or at least twice a week for two or more months, optionally further comprising administering tafasitamab at least once a month for subsequent months of treatment.

19.-20. (canceled)

21. The method of claim 14, wherein tafasitamab is administered on days 1, 4, 8, 15, and 22 of at least one 28-day cycle followed by administration of tafasitamab on days 1, 8, 15, and 22 of a second 28-day cycle followed by administration of tafasitamab on days 1 and 5 of one or more subsequent 28-day cycles.

22. The method of claim 15 wherein ibrutinib is administered orally in an amount from 300 mg to 700 mg/day or an amount of 400 mg/day to 600 mg/day.

23. (canceled)

24. The method of claim 14, where tafasitamab is delivered intraventicularly by lumbar puncture, optionally with an Ommaya reservoir.

25. (canceled)

26. The method of claim 1, wherein the CNS lymphoma is a primary lymphoma.

27. The method of claim 1, wherein CNS lymphoma is a secondary lymphoma.

28. The method of claim 1, wherein the patient has resistance to lenalidomide, pomalidomide, and/or ibrutinib.

29. The method of claim 1, wherein the CNS lymphoma is an intraocular lymphoma.

30. The method of claim 29, wherein tafasitamab is delivered by intraocular or intravitreal injection, optionally in an amount of about 0.1-0.5 mg/ml.

31. (canceled)

32. The method of claim 30, wherein tafasitamab is administered once a month.

33. A method of treating a neurodegenerative disease, the method comprising administered tafsitimab and a therapeutic compound in an amount to facilitate penetration of the therapeutic compound into the brain, optionally wherein the disease is multiple sclerosis.

34. (canceled)
